(19) United States
(12) Patent Application Publication
(10) Pub. No.: US 2013/0237988 A1
(43) Pub. Date: Sep. 12, 2013

(54) REVISION TOOL AND METHOD

(71) Applicant: Richard G. Mauldin, Erie, CO (US)

(72) Inventor: Richard G. Mauldin, Erie, CO (US)

(21) Appl. No.: 13/791,849

(22) Filed: Mar. 8, 2013

Related U.S. Application Data
(60) Provisional application No. 61/609,187, filed on Mar. 9, 2012.

(51) Int. Cl. A61B 17/92

(52) U.S. Cl. CPC A61B 17/92
USPC 606/84

(57) ABSTRACT

A revision tool may include a revision rod, crank and osteotome. The osteotome may include a shearing portion and a shank, and may be coupled to the revision rod via advancing device threads. The revision rod may be driven into an implant and engage the implant using an implant pin and implant threads. The osteotome may then be driven by the crank and positioned by the shank to sheer bone surrounding the implant. A slap hammer may then be used to remove the implant attached to the revision rod.

### CROSS REFERENCE TO RELATED APPLICATIONS

This patent application claims priority to U.S. Provisional Application No. 61/609,187, titled “REVISION TOOL AND METHOD”, filed on Mar. 9, 2012. This patent application may be related to one or more of the following patent applications: U.S. Patent Publication No. 2009/0259261, titled “SYSTEMS AND METHODS FOR THE FIXATION OR FUSION OF BONE AT OR NEAR A SACROILIAC JOINT”, filed on Dec. 4, 2008; U.S. Patent Publication No. 2010/0292738, titled “SYSTEMS AND METHODS FOR THE FIXATION OR FUSION OF BONE”, filed on Jul. 22, 2010; U.S. Patent Publication No. 2011/0087294, titled “SYSTEMS AND METHODS FOR THE FUSION OF THE SACRAL-ILIAC JOINT”, filed on Oct. 5, 2010; U.S. Patent Publication No. 2011/0118785, titled “APPARATUS, SYSTEMS, AND METHODS FOR ACHIEVING ANTERIOR LUMBAR INTERBODY FUSION”, filed on Dec. 6, 2010; and U.S. Patent Publication No. 2011/0118796, titled “SYSTEMS AND METHODS FOR THE FIXATION OR FUSION OF BONE”, filed on Jan. 18, 2011. Each of these references is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present invention relates generally to an implant revision tool. In various respects, the invention is directed to revision of a sacroiliac joint fusion device for connecting the sacrum to the ilium.

### BACKGROUND

Many types of hardware are available both for the fixation of bones that are fractured and for the fixation of bones that are to be fused. A fusion is an operation where two bones, usually separated by a joint, are allowed to grow together into one bone. The medical term for this type of fusion procedure is arthrodesis.

For example, lumbar fusion procedures have been used in the treatment of pain and the effects of degenerative changes in the lower back. An example of a lumbar fusion is a fusion in the S1-L5-L4 region in the spine.

Another example, the human hip girdle (see FIGS. 1 and 2) is made up of three large bones joined by two relatively immobile joints. One of the bones is called the sacrum and it lies at the bottom of the lumbar spine, where it connects with the L5 vertebra. The other two bones are commonly called “hip bones” and are technically referred to as the right ilium and the left ilium. The sacrum connects with both hip bones at the left and right sacroiliac joints (SI-Joint).

The SI-Joint functions in the transmission of forces from the spine to the lower extremities, and vice-versa. The SI-Joint has been described as a pain generator for up to 22% of lower back pain. To relieve pain generated from the SI Joint, sacroiliac joint fusion is typically indicated as a surgical treatment, e.g., for degenerative sacroiliitis, inflammatory sacroiliitis, iatrogenic instability of the sacroiliac joint, osteitis condensans ilii, or traumatic fracture dislocation of the pelvis.

Occasionally, after an implant device or implant fusion device has been implanted and secured into position, revision procedures are required to modify or remove the device that is impacted or has bony in-growth. Therefore, a method and apparatus are needed that allow safe and efficient removal of the implant device.

### SUMMARY OF THE DISCLOSURE

This application relates generally to an implant revision tool. In various respects, the application is directed to revision of a sacroiliac joint fusion device for connecting the sacrum to the ilium.

Some embodiments described herein provide for an orthopedic revision instrument for revising a bone implant including a revision rod having a proximal end, a distal end and a length extending between the proximal and distal ends, the revision rod includes an implant engagement portion having an implant pin and an implant thread, the implant pin adapted to enter an interior of the bone implant and the implant thread configured to mate with corresponding threads on the bone implant; and an osteotome for sheering bone and tissue surrounding the bone implant.

In some embodiments, the implant thread is located at the distal end of the revision rod. In other embodiments, the implant thread has a length between about 4 mm to about 6 mm. In further embodiments, the implant thread has a tapered distal end.

In some embodiments, the length of the revision rod is between about 200 mm and about 400 mm. Additionally, the revision rod may include a shaft member and an advancing device thread adjacent to the shaft member. In other embodiments, the revision rod also includes a revision thread located at the proximal end of the rod, the revision thread adapted to engage a slap hammer. In some variations, the revision thread includes a distal end that is adjacent to a proximal end of a gripping member on the rod. The gripping member may have a length between about 13 mm to about 15 mm. In some variations, the gripping member is knurled. In further embodiments, the advancing device thread extends from a distal end of the gripping member to a proximal end of the shaft. In any of the preceding embodiments, the advancing device thread has a length between about 80 mm to about 100 mm. The advancing device may include a diameter between about 6.5 mm to about 8 mm.

The orthopedic revision instrument may also include a crank configured to drive the osteotome into bone and tissue surrounding the implant. In other embodiments, the revision instrument includes a shank configured to align the osteotome with the bone implant. In any of the preceding embodiments, the orthopedic revision instrument may include or may couple to a slap hammer configured for removing the bone implant from bone and tissue surrounding the bone implant. The revision instrument may include a stopping member configured to engage a slap hammer at the proximal end of the rod.

In any of the preceding embodiments, the osteotome includes a shank portion and a sheering portion. The shank portion may include a lever socket for engaging a shank lever configured to align the osteotome with the bone implant. In other embodiments, the sheering portion includes a cutting edge at a distal end of the sheering portion, the cutting edge adapted to sheer bone as the osteotome is driven into bone. In some embodiments, the cutting edge is corrugated. In other embodiments, the cutting edge is tapered. In any of the preceding embodiments, the sheering portion comprises a triangular cross-section.

In any of the preceding embodiments, the bone implant removed may be removed from the sacroiliac joint.

Further embodiments provide a method for revising an implant. These methods include providing an orthopedic revision instrument comprising a revision rod and a osteotome coupled to the revision rod, the revision rod having an implant engagement portion for engaging a bone implant residing in a sacroiliac joint; inserting the revision rod into the sacroiliac joint and engaging the implant engagement portion with the bone implant, the implant engagement portion comprising a first threaded portion configured to mate with a second threaded portion on the bone implant; sheering bone in the sacroiliac joint that is surrounding the bone implant; and removing the bone implant from the sacroiliac joint.

In any of the preceding embodiments, inserting the revision rod includes driving the implant engagement portion into the implant. In some embodiments, the implant engagement portion comprises an implant pin adapted to fit inside the implant.

In any of the preceding embodiment, sheering bone includes driving the osteotome into bone of the sacroiliac joint.

In other embodiments, driving the osteotome includes rotating a T-crank to drive a sheering portion of the osteotome into bone in the sacroiliac joint. In some embodiments, the osteotome is driven over an exterior surface of the bone implant. In further embodiments, sheering bone includes cutting bone surrounding the bone implant with a cutting edge located at a distal end of the osteotome. Additionally, in some embodiments, removing the bone implant includes engaging a slap hammer to a proximal end of the revision rod to extract the bone implant from the sacroiliac joint.

Any of the preceding methods may include comprising aligning the osteotome with the bone implant. In some embodiments, aligning the osteotome further comprises engaging a lever to a shank portion of the osteotome to adjust the orientation of the osteotome.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-B illustrate exemplary embodiments of an implant device.

FIGS. 2-3 are, respectively, anterior and posterior anterior views of the human hip girdle comprising the sacrum and the hip bones (the right ilium, and the left ilium), the sacrum being connected with both hip bones at the sacroiliac joint.

FIGS. 4-6B are anatomic views showing, respectively, in pre-implanted perspective, implanted perspective, implanted anterior view, and implanted cranio-caudal section view, the implantation of three implant devices for the fixation of the SI-Joint using a lateral approach.

FIG. 7 illustrates an exemplary revision rod.

FIG. 8 illustrates an exemplary revision rod with an implant.

FIG. 9A is a perspective view of an exemplary revision tool.

FIG. 9B is a perspective view of an exemplary revision tool with a shank lever.

FIG. 10 is an enlarged distal end of an exemplary osteotome and an implant.

FIG. 11 is a perspective view of an exemplary revision tool with a slap hammer.

FIGS. 12A-C illustrate an exemplary method of revising an implant.

### DETAILED DESCRIPTION

Reference will now be made in detail to exemplary embodiments of the invention, examples of which are illustrated in the accompanying drawings. While the invention will be described in conjunction with the exemplary embodiments, it will be understood that they are not intended to limit the invention to those embodiments. On the contrary, the invention is intended to cover alternatives, modifications and equivalents, which may be included within the spirit and scope of the invention as described herein.

Various aspects of the present invention relate to a revision tool having a revision rod, crank and osteotome. In various embodiments, the revision tool may be used to remove an implant device from the sacroiliac joint. The revision tool discussed herein may also be used to remove an implant device from other bones within a human patient. For example, the revision tool may be used to remove an implant device from the lumbar region of the spine and other bones. As such, those of ordinary skill in the art will realize that exemplary embodiments related to removing an implant device from the sacroiliac joint are not limited to this joint, but rather set forth as examples.

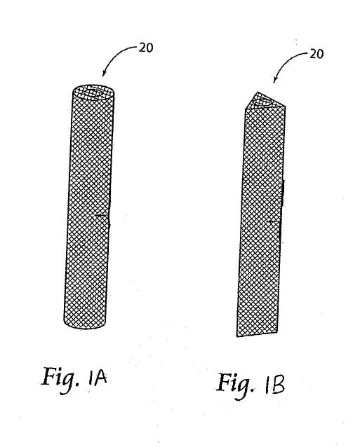

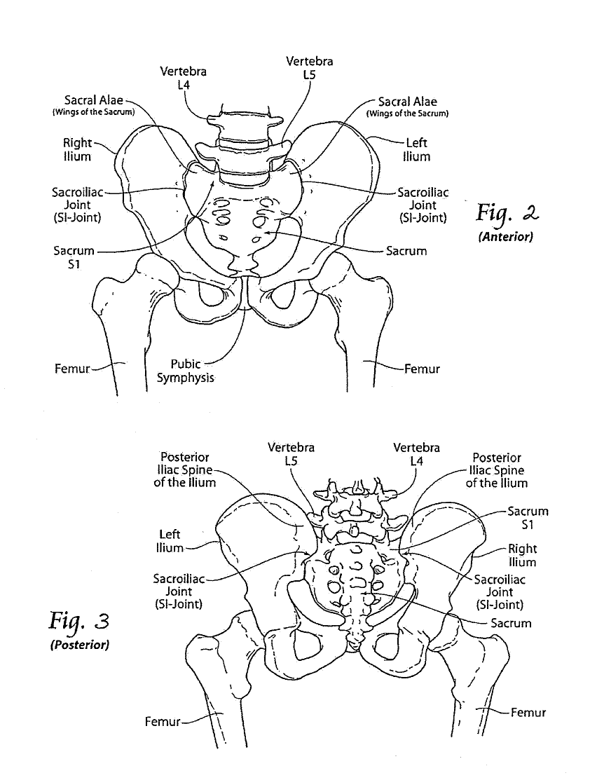

FIGS. 1A-B illustrate exemplary embodiments of an implant device. Elongated, stem-like implant devices 20 like that shown in FIGS. 1A-B make possible the fixation of the SI-Joint (shown in anterior and posterior views, respectively, in FIGS. 2 and 3) in a minimally invasive manner. Implant devices may have various shapes and have various cross-sectional geometries. For example, integrated implant 20 may have a generally curvilinear (e.g., round or oval) cross-section as illustrated in FIG. 1A or a generally rectilinear cross section (e.g., square or rectangular or triangular) as illustrated in FIG. 1B or combinations thereof. Implant devices 20 may be effectively implanted into the SI-Joint through the use of two alternative surgical approaches; namely, a lateral approach or a postero-lateral approach. Either procedure is desirably aided by conventional lateral and/or anterior-posterior (A-P) visualization techniques, e.g., using X-ray image intensifiers such as a C-arms or fluoroscopes to produce a live image feed that is displayed on a TV screen.

FIGS. 2-3 are, respectively, anterior and posterior views of the human hip girdle comprising the sacrum and the hip bones (the right ilium, and the left ilium), the sacrum being connected with both hip bones at the sacroiliac joint (SI-Joint).

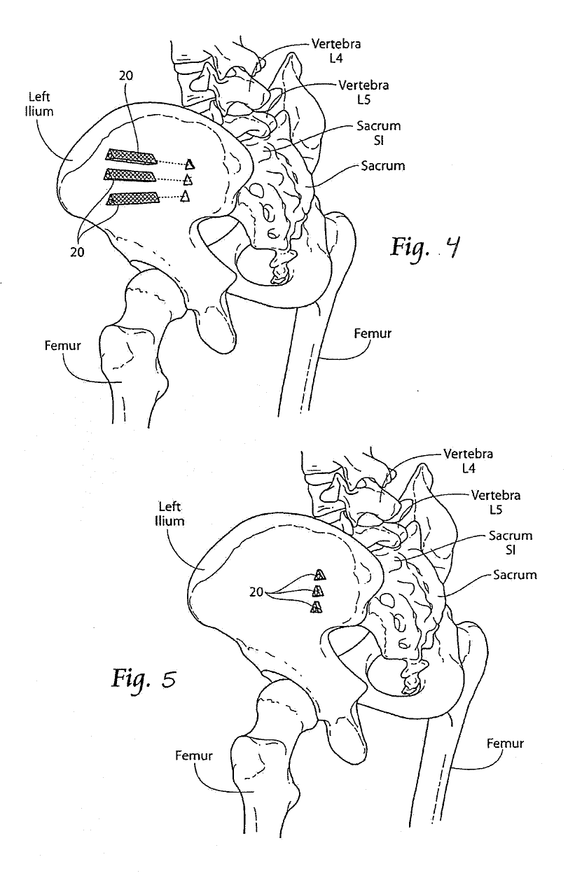

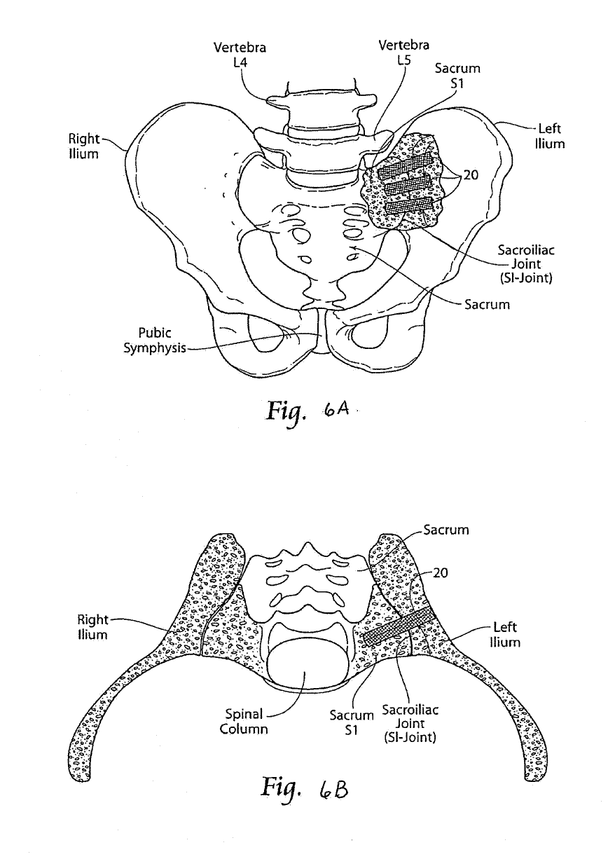

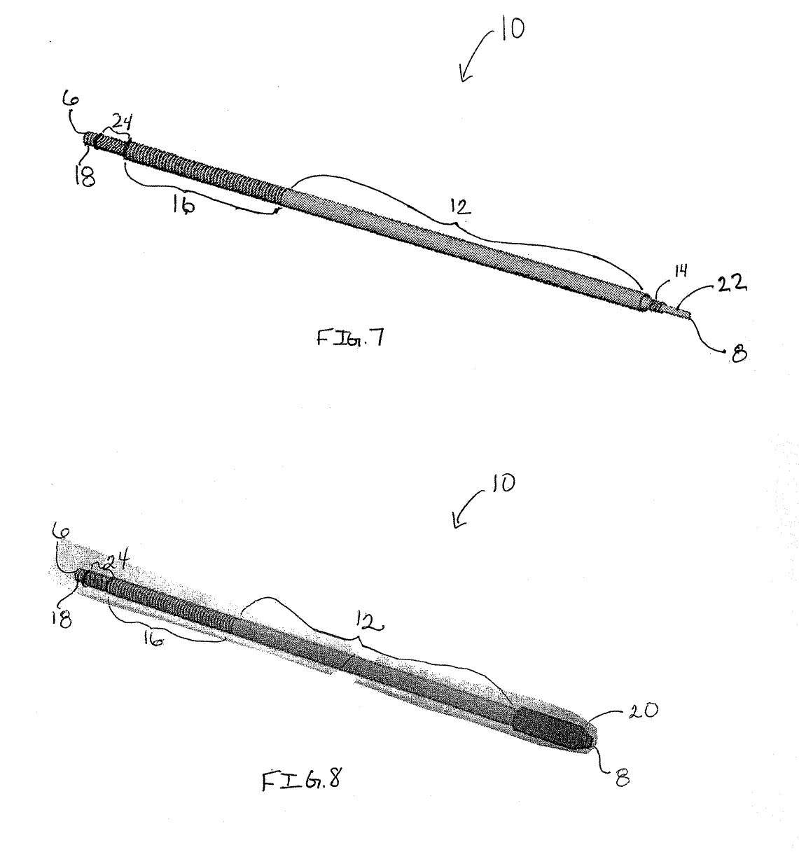

FIGS. 4-6B are anatomic views showing, respectively, in pre-implanted perspective, implanted perspective, implanted anterior view, and implanted cranio-caudal section view, the implantation of three implant devices for the fixation of the SI-Joint using a lateral approach. In one embodiment, one or more implant devices 20 are introduced laterally through the ilium, the SI-Joint, and into the sacrum. In the illustrated embodiment, three implant devices 20 are placed in this manner. Also in the illustrated embodiment, the implant devices 20 are triangular in cross section, but it should be appreciated that implant devices of other geometrical cross sections may be used.

FIG. 7 illustrates an exemplary revision rod. The exemplary revision rod 10 includes a proximal end 6, distal end 8, shaft 12, implant thread 14, advancing device thread 16, gripping member 24 and revision thread 18. The revision rod 10 may have a length extending from the proximal end 6 to the distal end 8 in the range of about 200 and 400 mm. In various embodiments, the length of revision rod 10 is about 335 mm. Implant thread 14 is located adjacent the distal end of shaft 12. When engaging implant 20, implant pin 22 may be inserted into implant 20 and implant threads 14 may engage a threaded portion inside of implant 20. The distal end of implant pin 22 may be rounded. Implant pin 22 may have a length in the range of about 14 to 16 mm and a diameter in the range of about 2 to 4 mm.

Revision thread 18 may be located at the proximal end 6 of revision rod 10. The revision thread 18 may have a length in the range of about 7 to 9 mm and a diameter of about 6.5 mm. In various embodiments, revision thread 18 engages a slap hammer. The distal end of revision thread 18 is adjacent to the proximal end of gripping member 24. Gripping member 24 may extend between revision thread 18 and the proximal end of advancing device thread 16. Gripping member 24 may have a length in the range of about 13 to 15 mm and a diameter of about 6.5 mm. In various embodiments, the gripping member is implemented as a light, straight knurl.

Advancing device thread 16 extends from the distal end of gripping member 24 to the proximal end of shaft 12. Advancing device thread 16 may have a length in the range of about 80 to 100 mm and a diameter in the range of about 6.5 to 8 mm.

Implant thread 14 may have a tapered distal end. Implant thread 14 may have a length of about 10 mm. The proximal diameter of the implant thread 14 may range from about 4 to 6 mm and the distal diameter may range from about 2 to 4 mm.

Additionally, although described as an implant pin and implant thread for mechanically engaging the implant, this is not intended to limit the manner in which the contemplated embodiments connect or couple to an implant. As can be appreciated, any suitable engagement mechanism or element can be used to couple the revision tool to an implant. For example, the revision tool may include an implant engagement portion adapted to form a friction-fit, interference-fit, press-fit, mated-fit, interlocking or locking-fit, keyed fitting etc. with the implant. Suitable mechanisms included threaded connections, cam locks/clamps, bayonet fittings, retaining or snap rings, ball-and-detect configurations, and/or mating/interference elements that are configured to engage and retain/lock/secure the implant to the revision tool.

FIG. 8 illustrates an exemplary revision rod with an implant. Implant 20 may be attached to distal end 8 of revision rod 10. Implant 20 may be positioned over implant pin 22 and may engage implant thread 14.

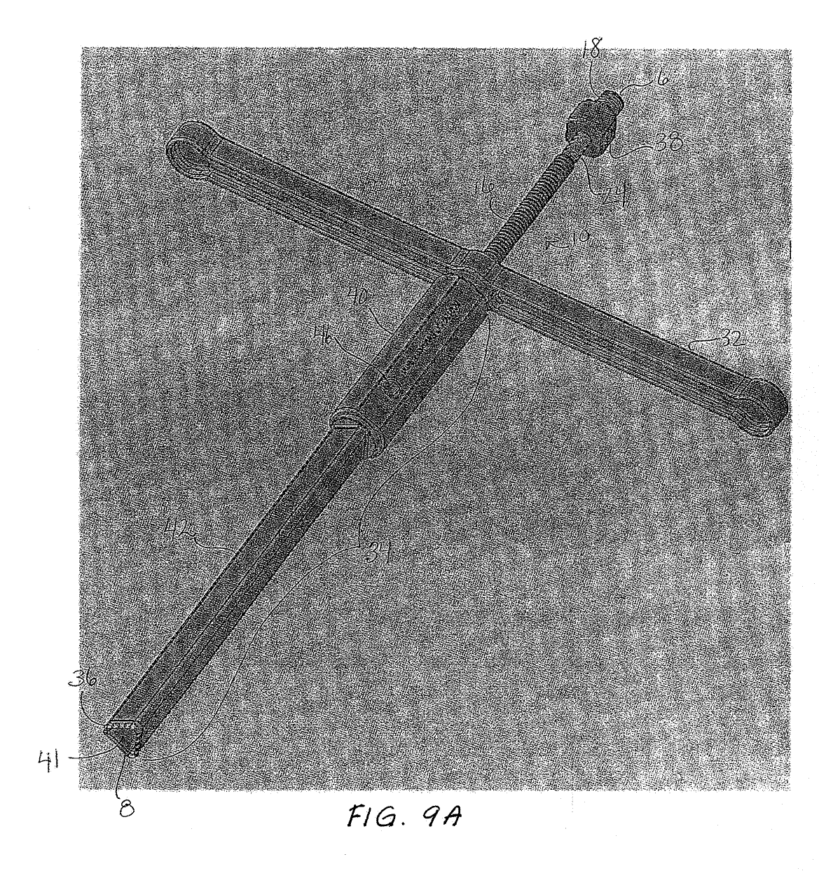

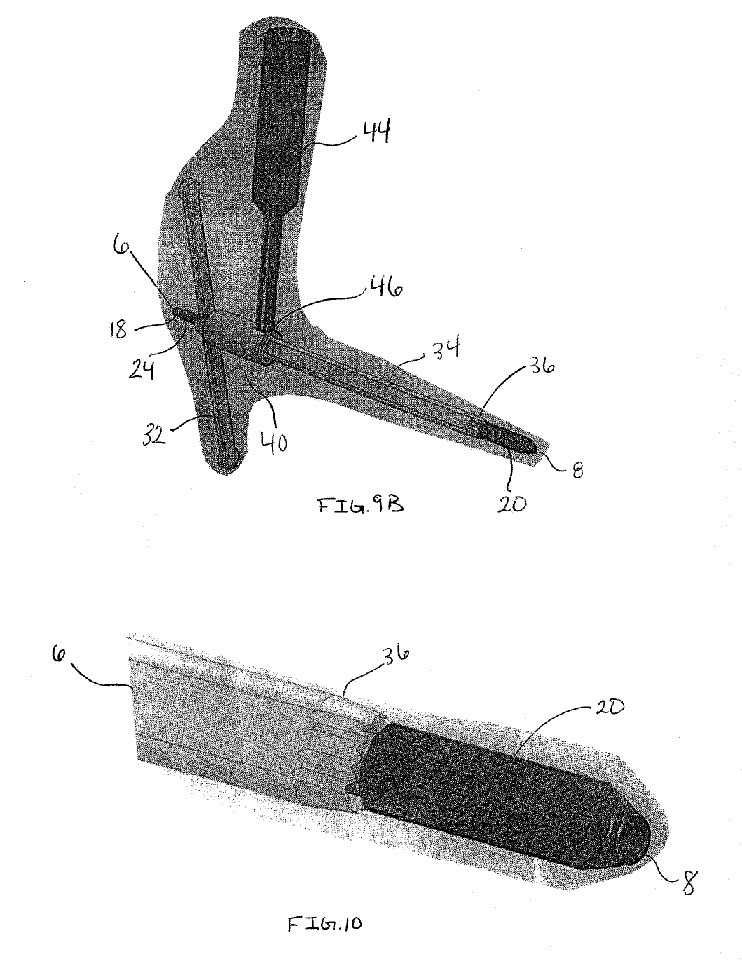

FIG. 9A is a perspective view of an exemplary revision tool. FIG. 9B is a perspective view of an exemplary revision tool with a shank lever. Revision tool 30 may include revision rod 10, T-crank 32, osteotome 34, cutting edge 36, and stopping member 38. T-crank 32 may engage advancing device threads 16. When T-crank 32 rotates around advancing device threads 16, osteotome 34 may be driven towards distal end 8 of the revision tool 30. Osteotome 34 may have a hollow portion 41 to allow revision rod 10 to be passed through the hollow portion 41.

Osteotome 34 may include a shank portion 40 and sheering portion 42. In various embodiments, shank portion 40 and sheering portion 42 may be formed as separate pieces. When formed as separate pieces, the distal end of shank 40 may also include a socket (not shown) to engage the proximal end of sheering portion 42. The shank portion 40 is located at or near the proximal end of the osteotome 34. Shank 40 may include lever socket 46 that may engage shank lever 44. Shank lever 44 may be used to rotationally position osteotome 34 to align with implant 20 (see FIG. 9B). Shank 40 may have a length in the range of about 83 to 85 mm and a diameter of about 18 mm.

Sheering portion 42 is located at or near the distal end of the osteotome 34. Sheering portion 42 may have a length in the range of about 190 to 210 mm. The distal end of sheering portion 42 may include cutting edge 36. Cutting edge 36 is configured to sheer bone as osteotome 34 is driven by T-crank 32. Sheering portion 42 may have a cross-section shape similar to the cross-section shape of implant 20. In the embodiment of FIG. 9A, sheering portion 42 has a triangular cross-section corresponding to a triangular shaped implant 20.

Stopping member 38 may engage the distal end of revision threads 18 and the proximal end of the gripping member 24. In various embodiments, the stopping member 38 is used with a slap hammer.

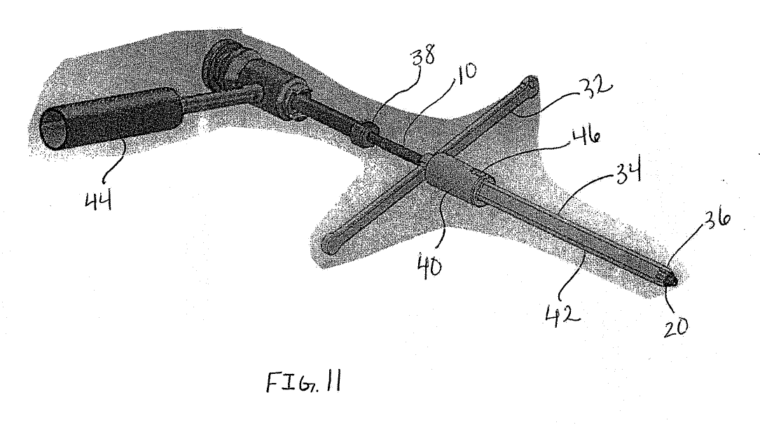

FIG. 10 is an enlarged distal end of an exemplary osteotome and an implant. Cutting edge 36 may be corrugated and may include a tapered distal end. Osteotome 34 is aligned by shank 40 such that cutting edge 36 is aligned with implant 20. As such, driving osteotome 34 causes cutting edge 36 to sheer bone and encompass implant 20 as osteotome 34 is driven by T-crank 32.

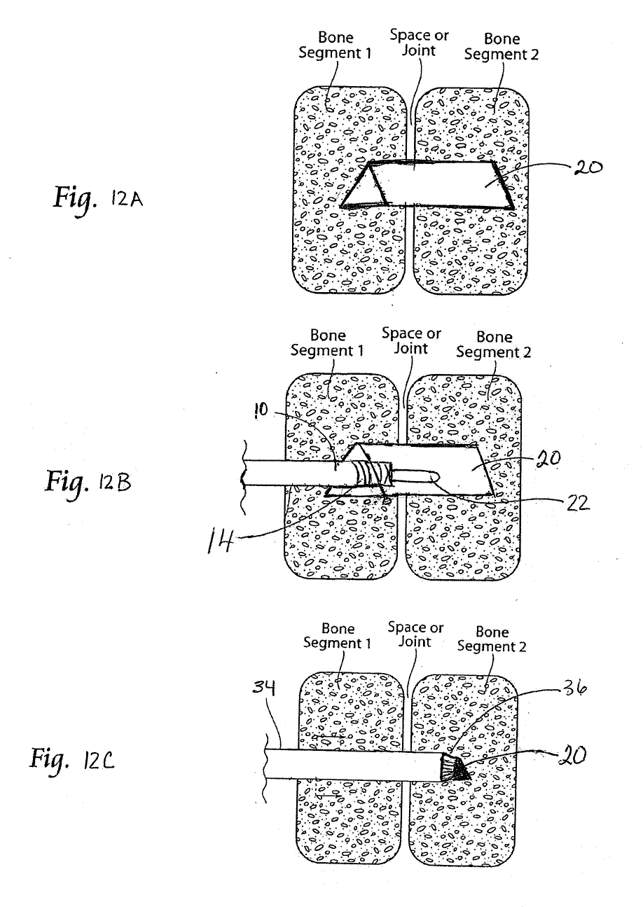

FIG. 11 is a perspective view of an exemplary revision tool with a slap hammer. Revision tool 30 may be coupled to slap hammer assembly 44. Slap hammer assembly 44 may engage the proximal end of revision thread 18, which is shown in FIG. 9B. A physician may use slap hammer assembly 44 in conjunction with stopping member 38 to remove implant 20 attached to the revision rod 10.

Revision tool 30 may be implemented effectively through the use of alternative surgical approaches. A lateral approach and a postero-lateral approach are discussed herein. Revision tool 30 may also be implemented using other surgical approaches. As such, those of ordinary skill in the art will realize that exemplary embodiments related to the revision tool are not limited to this type of surgical approach, but rather set forth as examples.

Before undertaking a lateral revision procedure, the physician identifies the implant device that is to be revised using conventional imaging techniques, e.g., using X-ray image intensifiers such as a C-arms or fluoroscopes to produce a live image feed that is displayed on a TV screen. In addition, local biopsies and tests may be performed if conditions such as an infection are suspected.

Aided by lateral, inlet, and outlet C-arm views, and with the patient lying in a prone position, the physician aligns the greater sciatic notches using lateral visualization to provide a true lateral position. An incision is made starting aligned with the posterior cortex of the sacral canal, followed by blunt-tissue separation to the implant(s) to be removed. From the lateral approach, the revision rod 10 is advanced to the lateral end of the implant 20 to be removed. The implant pin 22 is advanced into the implant guide hole and fastened to the implant 20 by turning the revision rod 10 clockwise until it is secured to the implant 20.

The set-up for a postero-lateral approach is generally the same as for a lateral approach. The physician identifies the implant device 20 that is to be revised. This exemplary procedure is typically performed with the patient lying in a prone position and may be aided by lateral and anterior-posterior (A-P) c-arms. The same surgical tools may be used except the insertion path now starts from the posterior iliac spine of the ilium, angles through the SI-Joint, and terminates in the sacral alae. The revision rod 10 is inserted from the posterior iliac spine of the ilium, angling through the SI-Joint, and terminating in the sacral alae, until the distal end of the revision rod 10 engages the proximal end of the implant structure 20.

FIGS. 12A-C illustrate an exemplary method of revising an implant. Implant device 20 may be revised using revision tool 30. FIG. 12A illustrates an implant device 20 installed between a first bone segment and a second bone segment. In various embodiments, the first bone segment is an ilium and the second bone segment is a sacrum. FIG. 12B illustrates a revision rod 10 engaging implant 20. Revision tool 30 may be positioned at the location of the implant. Implant pin 22 may inserted into the proximal end of the implant 20 until implant thread 14 reaches implant 20. Revision rod 20 is then rotated using gripping member 24 so that implant thread 14 engages the threading inside implant 20.

FIG. 12C illustrates osteotome 34 engaging implant 20. After revision rod 10 has engaged implant 20, osteotome 34 and T-crank 32 are positioned over revision rod 10 until advancing device thread 16 engages T-crank 32. T-crank 32 is then rotated to drive sheering portion 42 of the osteotome 34 into the patient and toward implant 20. Visualization techniques as described previously are used to determine the position of the revision tool 30 and implant 20 during the advancement of revision tool 30. Shank lever 44 may be attached to shank 40 and used to align cutting edge 36 with the proximal end of implant 20.

Once cutting edge 36 is aligned with implant 20, T-crank 32 is rotated further to drive sheering portion 42 and cutting edge 36 over the exterior surface of implant 20 removing bone and tissue growth. Slap hammer 44 or other removal device is then attached to revision thread 18 as previously shown and described to remove implant 20. If one or more implants are to be revised, the revision process is performed in the same manner for the remaining implants.

The revision tool described herein makes possible a revision of an implant device which is impacted or has bony in-growth. The design and configuration of the revision tool allow the osteotome to be aligned directly with the implant device to sheer bone from an implant device by the cutting edge as the osteotome is advanced. The revision tool optimizes bone removal while minimizing trauma to surrounding tissue. Additionally, the non-impact advancement of the osteotome minimizes the risk of over advancement of the revision tool and may be a safer option.

Additional details pertinent to the present invention, including materials and manufacturing techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the examples described herein, but only by the plain meaning of the claim terms employed.

What is claimed is:

1. An orthopedic revision instrument for revising a bone implant comprising:
a revision rod having a proximal end, a distal end and a length extending between the proximal and distal ends, the revision rod comprising an implant engagement portion having an implant pin and an implant thread, the implant pin adapted to enter an interior of the bone implant and the implant thread configured to mate with corresponding threads on the bone implant; and
an osteotome for sheering bone and tissue surrounding the bone implant.

2. The instrument of claim 1, wherein the length of the revision rod is between about 200 mm and about 400 mm.

3. The instrument of claim 1, wherein the implant thread is located at the distal end of the revision rod.

4. The instrument of claim 1, wherein the implant thread has a length between about 4 mm to about 6 mm.

5. The instrument of claim 1, wherein the implant thread has a tapered distal end.

6. The instrument of claim 1, wherein the revision rod further comprises a shaft member and an advancing device thread adjacent to the shaft member.

7. The instrument of claim 6, wherein the revision rod further comprises a revision thread located at the proximal end of the rod, the revision thread adapted to engage a slap hammer.

8. The instrument of claim 7, wherein the revision thread comprises a distal end that is adjacent to a proximal end of a gripping member on the rod.

9. The instrument of claim 8, wherein the gripping member has a length between about 13 mm to about 15 mm.

10. The instrument of claim 6, wherein the advancing device thread has a length between about 80 mm to about 100 mm.

11. The instrument of claim 6, wherein the advancing device has a diameter between about 6.5 mm to about 8 mm.

12. The instrument of claim 1, further comprising a crank configured to drive the osteotome into bone and tissue surrounding the implant.

13. The instrument of claim 1, further comprising a shank configured to align the osteotome with the bone implant.

14. The instrument of claim 1, further comprising a slap hammer configured for removing the bone implant from bone and tissue surrounding the bone implant.

15. The instrument of claim 1, wherein the bone implant is removed from the sacroiliac joint.

16. The instrument of claim 1, wherein the osteotome further comprises a shank portion and a sheering portion.

17. The instrument of claim 16, wherein the shank portion comprises a lever socket for engaging a shank lever configured to align the osteotome with the bone implant.

18. The instrument of claim 16, wherein the sheering portion comprises a cutting edge at a distal end of the sheering portion, the cutting edge adapted to sheer bone as the osteotome is driven into bone.

19. The instrument of claim 16, wherein the sheering portion comprises a triangular cross-section.

20. The instrument of claim 1, further comprising a stopping member configured to engage a slap hammer at the proximal end of the rod.

21. A method for revising an implant, comprising:
providing an orthopedic revision instrument comprising a revision rod and a osteotome coupled to the revision rod, the revision rod having an implant engagement portion for engaging a bone implant residing in a sacroiliac joint;
inserting the revision rod into the sacroiliac joint and engaging the implant engagement portion with the bone implant, the implant engagement portion comprising a first threaded portion configured to mate with a second threaded portion on the bone implant;
sheering bone in the sacroiliac joint that is surrounding the bone implant; and
removing the bone implant from the sacroiliac joint.

22. The method of claim 21, wherein inserting the revision rod further comprises driving the implant engagement portion into the implant.

23. The method of claim 21, wherein the implant engagement portion comprises an implant pin adapted to fit inside the implant.

24. The method of claim 21, wherein sheering bone further comprises driving the osteotome into bone of the sacroiliac joint.

25. The method of claim 24, wherein driving the osteotome further comprises rotating a T-crank to drive a sheering portion of the osteotome into bone in the sacroiliac joint.

26. The method of claim 24, wherein removing the bone implant further comprises engaging a slap hammer to a proximal end of the revision rod to extract the bone implant from the sacroiliac joint.
